(19) United States
(12) Patent Application Publication
(10) Pub. No.: US 2026/0294687 A1
(43) Pub. Date: Oct. 1, 2026

(54) PATIENT INTERFACE IMAGING SYSTEM

(71) Applicant: Alcon Inc., Fribourg (CH)

(72) Inventors: Steven T. Charles, Memphis, TN (US); David Abergel, Fort Worth, TX (US)

(21) Appl. No.: 19/576,346

(22) Filed: Mar. 24, 2026

Related U.S. Application Data
(60) Provisional application No. 63/780,583, filed on Mar. 31, 2025.

(51) Int. Cl. A61F 9/008; A61F 9/009

(52) U.S. Cl. CPC A61F 9/00825; A61F 9/009; A61F 2009/0087; A61F 2009/00872; A61F 2009/00887

(57) ABSTRACT

Embodiments herein provide an ophthalmic surgical system, including a laser delivery head, a patient interface configured to facilitate docking of the laser delivery head onto an eye for delivery of a laser to the eye, and a patient interface imaging system. The patient interface imaging system includes a plurality of scene cameras configured to capture images of the patient interface during docking of the laser delivery head onto the eye for display to a user on a graphical user interface (GUI). In embodiments, each of the plurality of scene cameras are arranged such that its optical axis is oriented at an oblique angle relative to a major axis of the laser delivery head, and such that each scene camera is arranged about 90°or more from another scene camera about the major axis of the laser delivery head.

### CROSS-REFERENCE TO RELATED APPLICATIONS

This application claims benefit of and priority to U.S. Provisional Patent Application No. 63/780,583, filed Mar. 31, 2025, which is hereby incorporated by reference in its entirety as though fully and complete set forth herein.

### INTRODUCTION

In certain ophthalmic procedures, laser pulses are directed by a laser system towards a patient's eye to interact with, e.g., create photodisruptions at, the lens and cornea of the eye. In certain such procedures, a patient interface can be affixed to the patient's eye (i.e., the eye can be “docked”) to limit movement between the laser system and the eye and to create a reliable optical interface between the laser system and the eye.

### SUMMARY

Embodiments of the present disclosure provide improved imaging systems for patient interfaces of laser surgical systems.

In certain embodiments, an ophthalmic surgical system is provided, the ophthalmic surgical system including: a chassis containing a laser source configured to generate a laser; a laser delivery head adjustably attached to the chassis and configured to deliver the laser to an eye; a patient interface, the patient interface comprising at least a first component attached to the laser delivery head and configured to facilitate docking of the laser delivery head onto the eye for delivery of the laser to the eye; a patient interface imaging system, the patient interface imaging system including: a first scene camera configured to capture images of the patient interface during docking of the laser delivery head onto the eye for display to a user on a graphical user interface (GUI); and a second scene camera configured to capture images of the patient interface during docking of the laser delivery head onto the eye, wherein: the first scene camera and the second scene camera are arranged such that optical axes of each of the first scene camera and the second scene camera are oriented at oblique angles relative to a major axis of the laser delivery head; and the first scene camera and the second scene camera are arranged about 90° or more from each other about the major axis of the laser delivery head.

In certain embodiments, an ophthalmic surgical system is provided, the ophthalmic surgical system including: a laser delivery device configured to deliver a laser to an eye of a patient; a patient interface attached to the laser delivery device and configured to facilitate docking of the laser delivery device onto the eye for delivery of the laser to the eye; and a patient interface imaging system, the patient interface imaging system comprising: a plurality of scene cameras configured to capture images of the patient interface during docking of the laser delivery device onto the eye for display on a graphical user interface (GUI), wherein each of the plurality of scene cameras is arranged such that an optical axis of the scene camera is oriented at an oblique angle relative to a major axis of the laser delivery device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a perspective view of an example ophthalmic surgical system for use with the patient interface imaging systems described herein, according to certain embodiments of the present disclosure.

FIG. 2 is a perspective view of another example ophthalmic surgical system for use with the patient interface imaging systems described herein, according to certain embodiments of the present disclosure.

FIG. 3A is an enlarged perspective view of a portion of an ophthalmic surgical system with a patient interface imaging system integrated therewith, according to certain embodiments of the present disclosure.

FIG. 3B is a schematic plan view of the ophthalmic surgical system of FIG. 3A with the patient interface imaging system integrated therewith, according to certain embodiments of the present disclosure.

FIGS. 4A-4B illustrate schematic side views of example arrangements of two or more scene cameras of a patient interface imaging system relative to a patient interface, according to certain embodiments of the present disclosure.

FIG. 5 illustrates a schematic top-down view of an arrangement of two or more scene cameras of a patient interface imaging system relative to a patient interface, according to certain embodiments of the present disclosure.

FIGS. 6A-6D illustrate various visuals that can be generated for a user based on images captured by various components of an ophthalmic surgical system during docking, according to certain embodiments of the present disclosure.

FIG. 7 illustrates an example robotic arm that can be used in combination with the ophthalmic surgical systems described herein, according to certain embodiments of the present disclosure.

FIG. 8 is a schematic plan view of various electronic components of the ophthalmic surgical systems described herein, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Certain ophthalmic surgical systems utilize a laser device that generates a pulsed laser beam to perform a surgical procedure on a patient's eye. For example, certain types of laser-assisted cataract surgery (LACS) systems generate a pulsed laser beam to create precise photodisruptions at specific locations on the patient's lens and cornea. A patient interface (PI) coupled to, or configured to be coupled to, the system is usually used to align the laser beam with the patient's eye during the procedure. As a result, the laser beam is properly aligned with the eye to create photodisruptions that precisely match a desired treatment pattern. Generally, the patient interface is affixed to the eye, or “docked” onto the eye, by vacuum suction or other similar method to secure the eye in place and to properly position the eye relative to the treatment pattern.

Typically, during the docking process, a patient is asked to gaze at a fixation light concentric with the laser beam. A surgeon then manually positions the laser device and/or patient interface onto the eye. However, ophthalmic surgical procedures are usually performed while the patient is laying on an operating bed, table, or other patient support, while the surgeon is disposed alongside and looking over the patient. Thus, to ensure proper alignment of the laser device and/or patient interface with the patient's eye during the docking process, the surgeon typically bends over to observe the docking at an oblique angle, otherwise the laser device and/or patient interface can obstruct the surgeon's view. This process is therefore non-ergonomic for the surgeon and can cause substantial discomfort, strain, or fatigue for the surgeon, who will thereafter perform the surgical procedure on the patient's eye.

To address this and other issues, embodiments described herein provide patient interface imaging systems for use during docking procedures for ophthalmic surgical procedures.

In certain embodiments, the patient interface imaging systems disclosed herein can be utilized with patient interfaces for laser-assisted cataract surgery (LACS) systems, femtosecond laser-assisted cataract surgery (FLACS) systems, laser-assisted in situ keratomileusis (LASIK) systems, laser epithelial keratomileusis (LASEK) systems, photorefractive keratectomy (PRK) systems, small incision lenticular extraction (SMILE) systems, smooth incision lenticular keratomileusis (SILK) systems, capsulectomy and/or iridectomy systems, selective laser trabeculoplasty systems, and/or other glaucoma surgical systems, retinal photocoagulation systems, etc. However, patient interfaces for other non-laser-assisted ophthalmic surgical systems are also contemplated, such as phacoemulsification or robotic cataract surgical systems.

Examples will now be described relative to the Drawings.

Note that, as described herein, a “distal” end, side, or portion of a component refers to the end, side, or the portion that is closer to the patient's body during the use thereof (e.g., at a far end away from the surgical device). On the other hand, a “proximal” end, side, or portion of the component refers to the end, side, or portion that is distanced further away from the patient's body (e.g., is closer to the surgical device).

As used herein, “about,” when used in conjunction with a numerical value, refers to ±5%, or ±10%, of the numerical value.

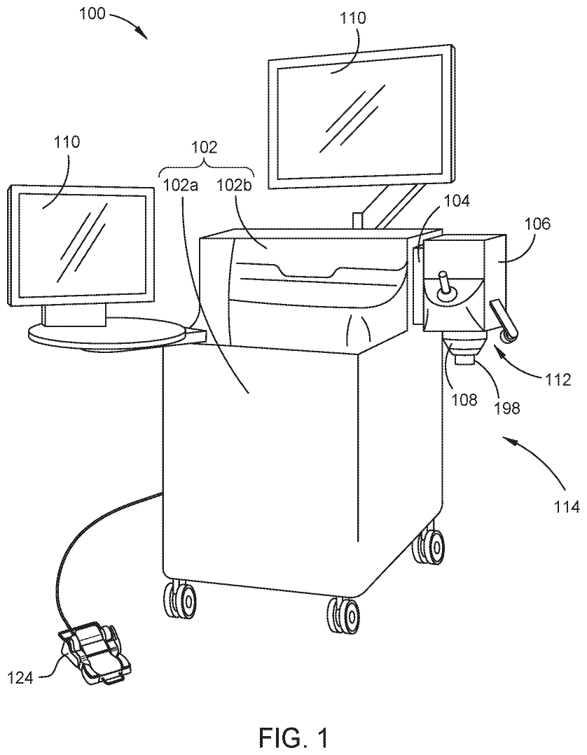

FIG. 1 illustrates a first example ophthalmic surgical system 100 that can be utilized with the patient interface imaging systems described herein, according to certain embodiments. In the illustrated example, the ophthalmic surgical system 100 is a laser-assisted cataract surgery (LACS) system, such as a femtosecond laser-assisted cataract surgery (FLACS) system; however, other types of ophthalmic surgical systems are also contemplated.

The ophthalmic surgical system 100 includes a lower chassis 102a and an upper chassis 102b (together referred to herein as a chassis 102), a laser delivery head 106 attached to the upper chassis 102b via a stationary laser delivery extension 104, a patient attachment module 108, and one or more graphical user interfaces (GUIs) 110. The lower and/or upper chassis 102a and 102b can each support one or more laser subsystems, imaging subsystems, interface subsystems, and/or other subsystems or computers of the ophthalmic surgical system 100. The laser delivery extension 104 and the laser delivery head 106 each include one or more optical devices, such as lenses, mirrors, beam splitters, beam combiners, projection systems, reflecting prisms, filters, fiber optics, and/or the like, for propagating laser light from the laser subsystems (and/or other types of light from, e.g., the imaging subsystems) to the patient attachment module 108.

The GUIs 110 can display and receive information and data to/from a user, such as a surgeon and/or other operating staff. In certain embodiments, a user can control one or more aspects of the ophthalmic surgical system 100 by providing inputs to the GUIs 110. In certain embodiments, the ophthalmic surgical system 100 further includes one or more other user input devices, such as a footswitch 124, a keyboard (not shown), and/or the like for controlling one or more aspects of the ophthalmic surgical system 100.

The patient attachment module 108 is disposed at a distal end 112 of the laser delivery head 106 and includes, or engages with, a patient interface 198 that is configured to dock onto a patient's eye to facilitate alignment of the laser delivery head 106 with the patient's eye during performance of a surgical procedure. In certain embodiments, although the laser delivery head 106 (and thus, the patient attachment module 108 and/or patient interface 198) is configured to be adjusted vertically and horizontally relative to the laser delivery extension 104 for docking to a patient's eye, the laser delivery head 106 is limited in rearrangement about a single patient side 114 of the ophthalmic surgical system 100. Thus, prior to performance of surgical procedures, a patient laying on an operating bed, table, or other patient support is positioned on the patient side 114 of the ophthalmic surgical system 100 with their head disposed below (e.g., distal to) the laser delivery head 106. The laser delivery head 106, and the patient attachment module 108 coupled thereto, are then adjusted vertically and, in some instances, horizontally, to facilitate docking of the laser delivery head 106 onto the patient's eye for performance of the surgical procedure. In other words, a height and/or lateral position of the laser delivery head 106 is adjusted to dock onto the patient's eye.

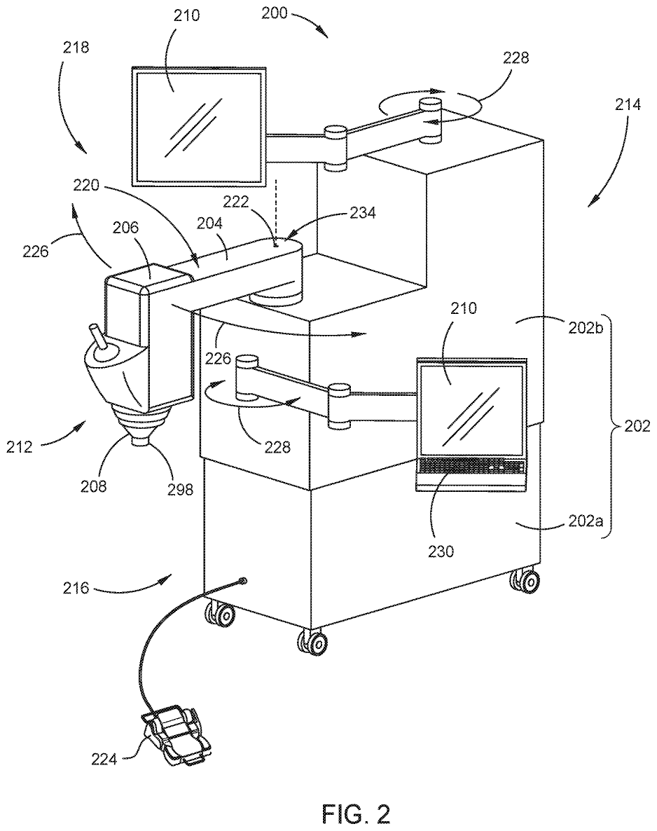

FIG. 2 illustrates a second example ophthalmic surgical system 200 that can be utilized with the patient interface imaging systems described herein, according to certain embodiments. Similar to the ophthalmic surgical system 100, the ophthalmic surgical system 200 is a laser-assisted cataract surgery (LACS) system, such as a femtosecond laser-assisted cataract surgery (FLACS) system; however, other types of ophthalmic surgical systems are also contemplated.

The ophthalmic surgical system 200 includes a lower chassis 202a and an upper chassis 202b (together referred to herein as a chassis 202), a rotatable laser delivery extension 204, a laser delivery head 206 attached to a distal end 220 of the laser delivery extension 204, a patient attachment module 208, and one or more graphical user interfaces (GUIs) 210. The lower and/or upper chassis 202a and 202b can each support one or more laser subsystems, imaging subsystems, interface subsystems, and/or other subsystems or computers of the ophthalmic surgical system 200. The laser delivery extension 204 and the laser delivery head 206 each include one or more optical devices, such as lenses, mirrors, beam splitters, beam combiners, projection systems, reflecting prisms, filters, fiber optics, and/or the like, for propagating laser light from the laser subsystems (and/or other types of light from, e.g., the imaging subsystems) to the patient attachment module 208.

The GUIs 210 can display and receive information and data to/from a surgeon and/or other operating staff. In certain embodiments, a user can control one or more aspects of the ophthalmic surgical system 100 by providing inputs to the GUIs 210. In certain embodiments, the ophthalmic surgical system 200 further includes one or more other user input devices, such as a footswitch 224, a keyboard 230, and/or the like for controlling one or more aspects of the ophthalmic surgical system 200.

The patient attachment module 208 is disposed at a distal end 212 of the laser delivery head 206 and includes, or engages with, a patient interface 298 that is configured to dock onto a patient's eye to facilitate alignment of the laser delivery head 206 with the patient's eye during performance of a surgical procedure. Unlike the ophthalmic surgical system 100, however, the laser delivery head 206 can be arranged along two or more sides of the ophthalmic surgical system 200 via rotation, or swinging, of the rotatable laser delivery extension 204 (in addition to adjustment of the laser delivery head 206 vertically and/or horizontally relative to the laser delivery extension 204). As indicated by arrows 226, the laser delivery extension 204 can be swiveled about a pivot point 222 at a proximal end 234 of the laser delivery extension 204 such that the distal end 220, where the laser delivery head 206 is disposed, can be arranged on at least a first side 214, second side 216, or third side 218 of the ophthalmic surgical system 200. And, once positioned at one of the first side 214, second side 216, or third side 218, the laser delivery head 206 and the patient attachment module 208 can be adjusted, vertically, for docking of the laser delivery head 206 to a patient's eye. Additionally, as indicated by arrows 228, the one or more GUIs 210 and/or other user input devices can also be adjustably arranged relative to the sides 214, 216, and 218 of the ophthalmic surgical system 200 to facilitate ergonomic viewing and/or use thereof by a user regardless of where the laser delivery head 206 is disposed.

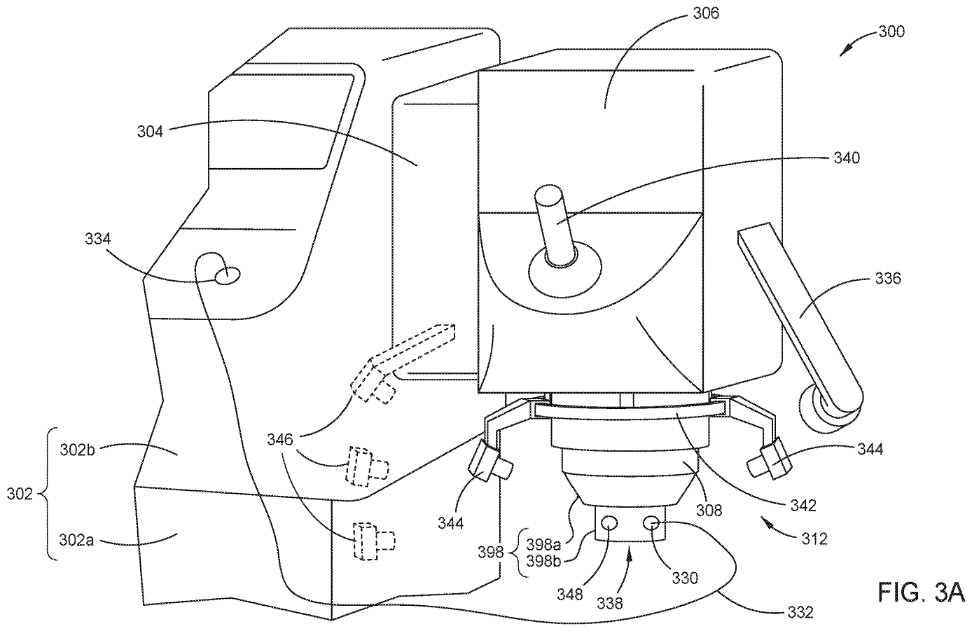

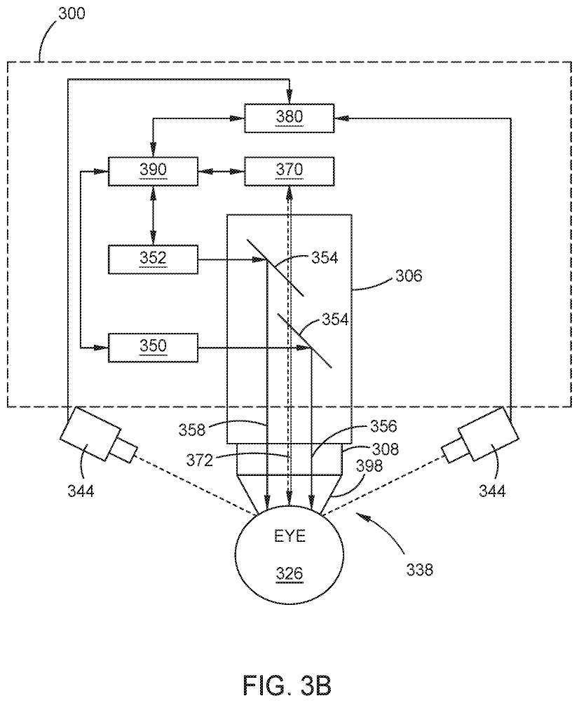

FIGS. 3A and 3B illustrate an example laser delivery head 306 of an ophthalmic surgical system 300 with a patient attachment module 308 and patient interface 398, according to certain embodiments described herein. FIG. 3A illustrates an enlarged side view of the laser delivery head 306, as well as other components of the ophthalmic surgical system 300. FIG. 3B illustrates a schematic cross-sectional plan view of the laser delivery head 306, as well as other components of the ophthalmic surgical system 300. Generally, the laser delivery head 306 is representative of the laser delivery heads 106 and 206, the patient attachment module 308 is representative of the patient attachment modules 108 and 208, the patient interface 398 is representative of the patient interfaces 198 and 298, and the ophthalmic surgical system 300 is representative of the ophthalmic surgical systems 100 and 200. Thus, although the arrangement of the laser delivery head 306 and ophthalmic surgical system 300 in FIG. 3A more closely resembles the laser delivery head 106 and ophthalmic surgical system 200, the illustration is merely representative and the description below is equally applicable to the laser delivery head 206 and ophthalmic surgical system 200.

With reference to FIG. 3A, the patient attachment module 308 is disposed at a distal end 312 of the laser delivery head 306, which couples to an upper chassis 302b of the ophthalmic surgical system 300 via a laser delivery extension 304. The laser delivery head 306 includes one or more optical devices for guiding laser light received from one or more laser subsystems of an ophthalmic surgical system to the patient attachment module 308. Meanwhile, the patient attachment module 308 includes, or engages with, the patient interface 398 that is configured to be docked, or attached, to an eye of a patient for alignment of the laser delivery head 306 with the patient's eye during performance of a surgical procedure, thereby assuring that the laser light received from the laser subsystems is delivered to the patient's eye.

Generally, the patient interface 398 may include a cone, a cup, a ring, or other similar structure that can be positioned against, or in contact with, the patient's eye during performance of a surgical procedure thereon. In certain embodiments, the patient interface 398 includes a multi-component system with two or more distinct components configured to interact, or interconnect, for docking of the patient interface 398 (and the laser delivery head 306) to the patient's eye. For example, in certain embodiments, the patient interface 398 includes a first component 398a, such as a cone that is fixedly attached to the patient attachment module 308, and a second component 398b, such as a ring or cup that is removably attached to the cone prior to, or during, docking of the patient interface 398 to the patient's eye. The separate second component 398b can, in certain examples, be placed onto the patient's eye and held in place via vacuum suction prior to lowering and attaching the first component 398a, the patient attachment module 308, and/or the laser delivery head 306 onto the second component 398b during docking of the patient's eye. For example, during docking, the first component 398a is translated down onto the second component 398b already disposed on the patient's eye to engage the two components 398a and 398b, and to dock the laser delivery head 306 onto the patient's eye.

In certain embodiments, one or more of the components of the patient interface 398 can be sterilizable and re-usable. In certain embodiments, one or more of the components of the patient interface 398 can be disposable and for one-time use. In certain embodiments, one or more of the components of the patient interface 398 can include one or more lenses or other optical devices, such as an applanation lens.

In certain embodiments, the patient interface 398, or another component of the patient attachment module 308, can be fluidly coupled with a vacuum source to facilitate vacuum suction of the patient's eye to the patient interface 398 for at least the duration of the surgical procedure. Such a vacuum source can be disposed within the upper chassis 302b or a lower chassis 302a (together referred to as the chassis 302) of the ophthalmic surgical system 300. Accordingly, one or more components of the patient interface 398 can include a port 330 configured to receive a fluid line 332, which can extend from the patient interface 398 and through a corresponding port 334 in, for example, the upper chassis 302b to couple with the vacuum source (not shown).

In certain embodiments, one or more components of the patient interface 398 and/or the patient attachment module 308 includes a pressure sensor 348 integrated therewith. The pressure sensor 348 can be configured to measure an amount of opposing force(s) applied against the patient interface 398 by, for example, the patient's eye during docking and/or during performance of a surgical procedure. The measured force(s) can then be displayed on a GUI of the ophthalmic surgical system 300 for viewing by the user to identify instances when too much pressure (e.g., beyond a target or safe threshold) is being applied to the patient's eye. In certain embodiments, the ophthalmic surgical system 300 can further determine a likelihood of corneal folding of the patient's eye based on measured force(s), and can display the determination on the GUI to notify the user of the risk. Generally, the pressure sensor 348 can include any suitable type of force sensor, such as a force torque sensor or strain gauge.

In certain embodiments, an illuminator 336 is fixedly or adjustably attached to an exterior of the laser delivery head 306 and is configured to transmit illumination light toward a distal end 338 of the patient attachment module 308 where the patient interface 398 is disposed or configured to attach to. The illumination light transmitted by the illuminator 336 can illuminate the patient interface 398 and/or an area adjacent thereto during docking and/or during a surgical procedure to provide improved visualization thereof by a user. In certain embodiments, the illuminator 336 itself comprises an illumination source, such as one or more light-emitting diodes (LEDs), lamps, or other light-emitting devices. In certain embodiments, the illuminator 336 transmits light generated by an illumination source found elsewhere in the ophthalmic surgical system 300, such as in the chassis 302.

In certain embodiments, a position of the laser delivery head 306, for example, relative to the laser delivery extension 304, can be at least partially adjusted by a user via a joystick 340 or other user control device. For example, once a patient's eye is positioned below (e.g., distal to) the patient attachment module 308 and/or patient interface 398, the user can manipulate the joystick 340 to lower the laser delivery head 306 and translate the patient interface 398 onto the patient's eye for docking. In certain embodiments, the joystick 340 can further be configured to adjust a position of the laser delivery extension 304 and/or other components of the ophthalmic surgical system 300.

As further shown, one or more scene cameras 344 of a patient interface imaging subsystem 380 (shown in FIG. 3B) are attached to at least one of the patient attachment module 308, laser delivery head 306, laser delivery extension 304, chassis 302, and/or other components of the ophthalmic surgical system 300. The one or more scene cameras 344 are configured to capture images of at least the distal end 338 of the patient attachment module 308, the patient interface 398, and/or the patient's eye during docking. Such images can then be presented on a display of the ophthalmic surgical system 300 for viewing by a user, such as a surgeon or other operating staff, to assist in the docking of the laser delivery head 306 to the patient's eye, without the surgeon or operating staff member having to physically bend over to observe the docking process. As utilized herein, the term, “images,” can refer to static images or videos. Videos can be captured in real-time or near real-time. For example, in certain embodiments, the devices and systems described herein capture live video stream(s) for display to the user (e.g., on a graphical user interface (GUI) or other display device) to assist the user in a docking process. The images can be either two-dimensional (2D) or three-dimensional (3D).

In certain embodiments, one or more of the scene cameras 344 are attached to a component of the ophthalmic surgical system 300 in a fixed position and orientation. For example, one or more scene cameras 344 can be fixedly positioned and oriented on the laser delivery head 306 and/or laser delivery extension 304 such that an optical axis of the scene camera(s) 344 is always directed toward the distal end 338 of the patient attachment module 308 and/or the patient interface 398, even with movement of the laser delivery head 306 and/or laser delivery extension 304. In certain embodiments, one or more of the scene cameras 344 are fixed in position, but are adjustable in orientation. For example, one or more scene cameras 344 can be tilted, or angled, to adjust a direction of the optical axis of the scene camera(s) 344, but cannot be translated from one position to another. In certain embodiments, one or more of the scene cameras 344 are adjustable in position and orientation. In certain embodiments, one or more of the scene cameras 344 are adjustable in position, but not in orientation.

In embodiments where one or more of the scene cameras 344 are adjustable in position, the scene camera(s) 344 can be slidably or fixedly attached to a rail 342 or other feature coupled to the patient attachment module 308, laser delivery head 306, and/or other components of the ophthalmic surgical system 300. In such embodiments, the scene camera(s) 344 can be translated around the periphery of the patient attachment module 308 along the rail 342 (and in certain embodiments, adjusted in tilt/angle relative to the distal end 338 of the patient attachment module 308), or the rail 342 can be rotated around the patient attachment module 308, to obtain images from one or more positions around the periphery of the patient attachment module 308 and/or the patient interface 398. In some embodiments, the scene camera(s) 344 may be spaced circumferentially about the patient attachment module 308, the patient interface 398, and/or other components in a non-uniform manner, with a larger space between two of the scene cameras 344. In these and other embodiments, the rail 342 and/or the scene cameras 344 may be rotated such that the larger space is oriented in front of the surgeon, giving the surgeon more room to operate without interference of the scene cameras 344. In certain embodiments, the scene camera(s) 344 can be attached to a robotic arm, as further described below.

In the illustrated embodiment, two scene cameras 344 are shown attached to a rail 342 disposed around/attached to the patient attachment module 308. However, the ophthalmic surgical system 300 can include any number of scene cameras 344 for the patient interface imaging subsystem 380, such as more or less than two scene cameras 344. Further, the scene cameras 344 can be attached or integrated elsewhere on the ophthalmic surgical system 300, such as directly onto the patient attachment module 308, the laser delivery extension 304, the upper chassis 302b, and the lower chassis 302a, as represented by phantom scene cameras 346 in FIG. 3A, or onto other components of the ophthalmic surgical system 300 such as the laser delivery head 306.

Turning to FIG. 3B, various components of the ophthalmic surgical system 300 are schematically illustrated. As shown, the ophthalmic surgical system 300 includes a first laser subsystem 350, a second laser subsystem 352, a treatment imaging subsystem 370, and the patient interface imaging subsystem 380.

Generally, each of the first laser subsystem 350 and the second laser subsystem 352 include one or more laser sources, sensors, detectors, light sources, lenses, mirrors, beam splitters, beam combiners, projection systems, reflecting prisms, dispersing devices, filters and thin films, fiber optics, and/or other associated devices for performing one or more laser-based functions of the ophthalmic surgical system 300. For example, one of the first laser subsystem 350 and the second laser subsystem 352 can generate and transmit a treatment laser beam for treating a condition of a patient's eye 326 (hereinafter, “the eye 326”), while the other of the first laser subsystem 350 and the second laser subsystem 352 can generate and transmit an aiming laser beam to facilitate aiming of the treatment laser beam toward target tissues within the eye 326. The laser beams generated by each of the first laser subsystem 350 and the second laser subsystem 352 can be transmitted through at least a portion of the laser delivery head 306 and the patient attachment module 308, and/or the chassis 302 in FIG. 3A, via one or more optical devices 354 (e.g., lenses, mirrors, beam splitters, beam combiners, etc.). Such laser beams, and/or other types of light propagated by the first laser subsystem 350 and/or the second laser subsystem 352, can exit/enter the ophthalmic surgical system 300 (e.g., to/from the eye 326) through the patient interface 398 at the distal end 338 of the patient attachment module 308. The laser beams and/or other types of light propagated by the first laser subsystem 350 and/or the second laser subsystem 352 are transmitted to/from the eye 326 along treatment optical axes 356 and 358, respectively, which can be parallel or non-parallel to each other.

In certain embodiments, the first laser subsystem 350 and the second laser subsystem 352 share one or more optical devices 354. For example, in such embodiments, the laser beams generated by the first laser subsystem 350 and the second laser subsystem 352 can be combined utilizing the shared optical devices 354 before exiting the ophthalmic surgical system 300. In certain embodiments, the first laser subsystem 350 and the second laser subsystem 352 are integrated into a single laser subsystem 350 or 352.

The treatment imaging subsystem 370 includes one or more light sources (e.g., for illumination of the eye 326), sensors, detectors, cameras, lenses, mirrors, beam splitters, beam combiners, projection systems, reflecting prisms, dispersing devices, filters and thin films, fiber optics, and/or other associated devices for performing one or more imaging-based functions of the ophthalmic surgical system 300. In certain embodiments, the treatment imaging subsystem 370 includes an optical coherence tomography (OCT) system such as a swept source-OCT (SS-OCT) system, an OCT retina and/or anterior segment imaging system, a low-coherence interferometry system, a conventional or wide-angle fundus camera system, a digital fundus camera system, a fundus autofluorescence (AF) and/or multispectral imaging system, an ultrasound system, or other type of imaging systems. Generally, the treatment imaging subsystem 370 is configured to receive, and/or to generate and transmit, light for imaging the eye 326 during a surgical procedure involving the utilization of the first laser subsystem 350 and/or the second laser subsystem 352. The light propagated by the imaging subsystem 370 can be transmitted through at least a portion of the laser delivery head 306 and the patient attachment module 308, and/or the chassis 302 via the one or more optical devices 354 (e.g., lenses, mirrors, beam splitters, beam combiners, etc.). Such light can exit/enter the ophthalmic surgical system 300 (e.g., to/from the eye 326) through the patient interface 398 at the distal end 338 of the patient attachment module 308. The light propagated by the treatment imaging subsystem 370 are transmitted to/from the eye 326 along imaging optical axis 372, which can be parallel to one or both of treatment optical axes 356 and 358. In certain embodiments, the treatment imaging subsystem 370 shares one or more optical devices 354 with the first laser subsystem 350 and/or the second laser subsystem 352 share one or more optical devices 354.

The patient interface imaging subsystem 380 includes one or more scene cameras 344 (two are illustrated in FIG. 3B), sensors, detectors, fiber optics, and/or other associated devices for capturing, transmitting, processing, and displaying images of the patient attachment module 308 and/or the patient interface 398 attached thereto and the eye 326 during docking and/or performance of a surgical procedure. In certain embodiments, the patient interface imaging subsystem 380 is integrated with the treatment imaging subsystem 370 in a single imaging subsystem 370 or 380, as both subsystems can be configured to perform similar imaging/visualization functions (though from different viewpoints and/or axes) and thus, can share one or more imaging devices.

Generally, the scene cameras 344 are configured to capture images of an area adjacent to the patient interface 398 or the patient attachment module 308, and relay such images to other components of the patient interface imaging subsystem 380 (or other subsystem) for further processing and/or display. In certain embodiments, the scene cameras 344 are wiredly connected to the other components of the patient interface imaging subsystem 380; in certain embodiments, the scene cameras 344 are wirelessly connected to the other components of the patient interface imaging subsystem 380. The scene cameras 344 can be configured to transmit image data to other components of the patient interface imaging subsystem 380 via high-speed Mobile Industry Processor Interface (MIPI) transmission protocols, High-Definition Multimedia Interface (HDMI) transmission protocols, or Universal Serial Bus (USB) transmission protocols.

Generally, one or more of the scene cameras 344 include small-form-factor cameras or image sensors, such as single-chip cameras with image signal processors (ISP). In certain embodiments, one or more of the scene cameras 344 include complementary metal oxide semiconductor (CMOS) cameras, or other active-pixel image sensors, such as front-illuminated CMOS cameras and/or back-illuminated CMOS cameras. In certain embodiments, one of more of the scene cameras 344 are configured to capture and produce black-and-white outputs; in certain embodiments, one or more of the scene cameras 344 are configured to capture and produce colored outputs. In certain embodiments, one or more of the scene cameras 344 include infrared (IR) cameras, such as red-green-blue-infrared (RGB-IR) cameras with a color filter array (CFA) having dedicated pixels for visible and IR light, to enable image capture in both low-light and well-lit environments. In such embodiments, the IR functionality of the scene cameras 344 can be beneficial for photophobic, or light-sensitive, patients, as the scene cameras 344 can be used without the patient attachment module 308, the patient interface 398, and/or the patient's eye 326 being illuminated with bright light.

In certain embodiments, one or more of the scene cameras 344 include molded polymer optics, or plastic optics. As compared to traditional glass optics, plastic optics are low-weight and provide greater design flexibility, as plastic optics can be produced in a wide range of shapes and microstructures, including aspheric optical surfaces. However, glass optics are also contemplated for the scene cameras 344. In certain embodiments, one or more of the scene cameras 344 include thin and aberration-free liquid lenses composed of cells containing an optical-grade liquid material (e.g., water and/or oil) that can change shape in response to electrical input. Upon receipt of electrical control signals, such lenses can be adjusted in curvature radius and thus, focal length, while remaining in place in the scene cameras 344. In certain embodiments, one or more of the scene cameras 344 include optics designed to provide a wide field-of-view (FOV) for the scene camera(s) 344, such as wide-angle lenses and fisheye-type lenses.

In certain embodiments, one or more of the scene cameras 344 include optics designed to have an adjustable magnification. In certain embodiments, one or more of the scene cameras 344 include telecentric optics designed to have a constant magnification regardless of the object's distance or location in a field-of-view (FOV) of the scene camera(s) 344.

As further shown in FIG. 3B, each of the first laser subsystem 350, the second laser subsystem 352, the imaging subsystem 370, and/or the patient interface imaging subsystem 380 is in data communication with at least one controller 390 of the ophthalmic surgical system 300. Generally, the controller 390 includes a processor and associated memory, and can be utilized to control one or more components of the various subsystems in performing their associated one or more functions. In certain embodiments, each of the first laser subsystem 350, the second laser subsystem 352, the imaging subsystem 370, and/or the patient interface imaging subsystem 380 is in data communication with a common, shared controller 390. In other embodiments, one or more of the first laser subsystem 350, the second laser subsystem 352, the imaging subsystem 370, and/or the patient interface imaging subsystem 380 is in data communication with a distinct controller 390 dedicated to such subsystem.

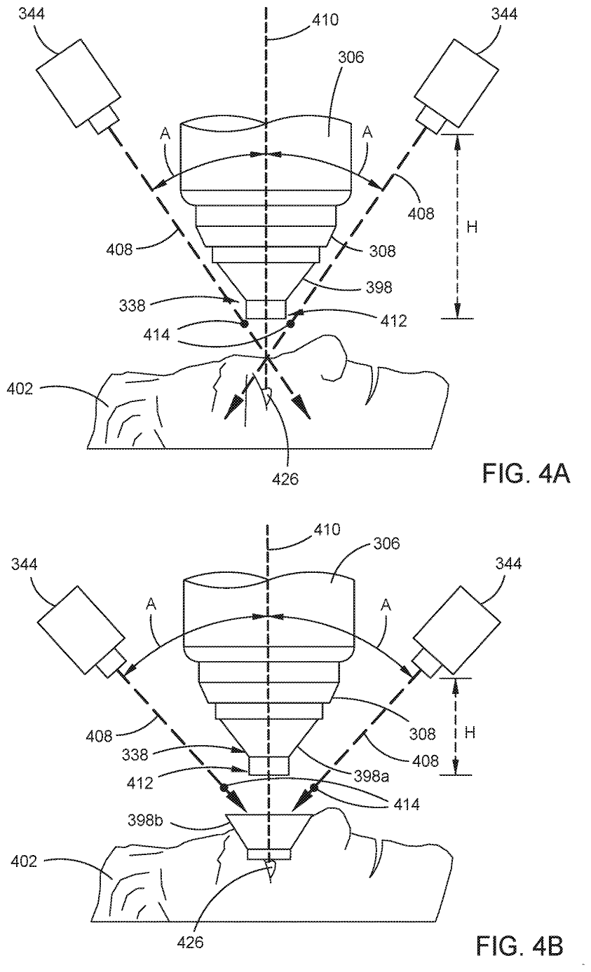

FIGS. 4A and 4B illustrate example arrangements of the scene camera(s) 344 of the patient interface imaging subsystem 380 relative to different types of the patient interface 398, according to certain embodiments described herein. In both of FIGS. 4A and 4B, schematic side views of the scene camera(s) 344, the patient interface 398, and a patient 402 are shown.

Referring to FIG. 4A, the patient interface 398 is a single or multi-component device configured to dock (e.g., attach) onto an eye 426 of the patient 402 for performance of an ophthalmic surgical procedure. In the illustrated embodiments, all of the components of the patient interface 398 are fixedly or removably attached to the distal end 338 of the patient attachment module 308 and/or the laser delivery head 306 prior to docking to the patient 402, such that when the laser delivery head 306 and the patient attachment module 308 are lowered during docking, the patient interface 398 contacts the eye 426 for the first time after already being attached to the laser delivery head 306.

Referring now to FIG. 4B, the patient interface 398 is a multi-component device having the first component 398a and the second component 398b. In the illustrated embodiments, prior to docking, the first component 398a is fixedly or removably attached to the patient attachment module 308 and/or laser delivery head 306, while the second component 398b is placed onto the eye 426. In certain embodiments, the second component 398b can be held in place on the eye 426 via application of vacuum suction from a vacuum source fluidly coupled to the second component 398b, as described above with reference to FIG. 3A. During docking, the first component 398a is lowered down onto the second component 398b already disposed on the eye 426 to couple the two components 398a and 398b together, which docks the laser delivery head 306 onto the eye 426 via the patient interface 398.

Referring now to both FIGS. 4A and 4B, one or more scene cameras 344 are arranged around the patient attachment module 308 and are oriented toward the patient interface 398. As noted above, the scene cameras 344 can be directly or indirectly attached to any suitable components of the ophthalmic surgical system 300, such as the patient attachment module 308, laser delivery head 306, laser delivery extension 304, chassis 302, etc., and pointed towards the distal end 338 of the patient attachment module 308 and/or the patient interface 398.

Generally, each of the scene cameras 344 is oriented such that an optical axis 408 of the scene camera 344 is disposed at an angle A relative to a major vertical axis 410 of the laser delivery head 306, and/or relative to the treatment optical axes 356 and/or 358 and/or the imaging optical axis 372 of FIG. 3B. In certain embodiments, one or more of the scene cameras 344 is oriented such that the angle A is an oblique angle. In certain other embodiments, one or more of the scene cameras 344 is oriented such that the angle A is a right angle. In certain embodiments, two or more (or all) of the scene cameras 344 of the patient interface imaging subsystem 380 are oriented at different angles A. In certain embodiments, two or more (or all) of the scene cameras 344 of the patient interface imaging subsystem 380 are oriented at a same angle A.

In certain embodiments, one or more of the scene cameras 344 is oriented such that the optical axis 408 of the scene camera 344 passes, or extends, just below (e.g., distal to) a distal end 412 of one or more components of the patient interface 398 that are attached to the patient attachment module 308 prior to/during docking. For example, in FIG. 4A, the distal end 412 is a distal end of the entire patient interface 398; in FIG. 4B, the distal end 412 is a distal end of the component 398a of the patient interface 398 attached to the patient attachment module 308. Generally, such an orientation provides ample view of both the patient interface 398 and the eye 426 to the surgeon or other surgical staff during the docking process. In certain embodiments, however, one or more of the scene cameras 344 is oriented such that the optical axis 408 of the scene camera 344 is aligned with (e.g., passes through) the distal end 412 of the one or more components of the patient interface 398..

In certain embodiments, one or more of the scene cameras 344 has a fixed focus at a focal point or plane 414 relative to the patient interface 398 and/or patient attachment module 308. Generally, the focal point or plane 414 can be disposed just below the distal end 412. However, other positions for the focal point or plane 414 are also contemplated. In certain embodiments, two or more of the scene cameras 344 of the patient interface imaging subsystem 380 have a fixed focus at focal points or planes 414 disposed at the same height along the major vertical axis 410. In certain embodiments, two or more of the scene cameras 344 of the patient interface imaging subsystem 380 have a fixed focus at focal points or planes 414 disposed at different heights along the major vertical axis 410. In certain embodiments, using multiple scene cameras 344 fixed at different focal points or planes 414 enables the capture and generation of three-dimensional (3D) images of the patient interface 398 and eye 426 during docking. In some embodiments, the scene cameras 344 may utilize an automatic focusing feature such that the eye 426 and/or the second component 398b remains in focus as the laser delivery head 306 is lowered towards the eye 426 during a docking procedure.

Each of the scene cameras 344 is arranged at a height H relative to the distal end 412 of the patient interface 398. In certain embodiments, two or more (or all) of the scene cameras 344 of the patient interface imaging subsystem 380 are arranged at a same height H. In certain embodiments, two or more (or all) of the scene cameras 344 of the patient interface imaging subsystem 380 are arranged at different heights H. In certain embodiments, arranging multiple scene cameras 344 at different heights H facilitates the capture and generation of 3D images of the patient interface 398 and eye 426 during docking.

Generally, 3D images can be formed by capturing images of the same “scene” (e.g., the patient interface 398 and the eye 426) from multiple different perspectives relative to the scene, or at different focal lengths relative to the scene, and then processing and combining these images to create a depth illusion. In ophthalmic surgical system 300, such processing and combining can be performed by, e.g., controller 390, which is in data communication with at least the patient interface imaging subsystem 380. Controller 390, in certain embodiments, is configured to extract depth information from two-dimensional (2D) images captured by scene cameras 344 and recreate 3D representations therefrom.

In certain embodiments, 2D images captured by multiple scene cameras 344 disposed at different heights H, thus providing different perspectives or angles of the patient interface 398 and eye 426 (e.g., the “scene”), can be used to construct 3D images using photogrammetry. In other words, the 2D images can be combined by controller 390 using image processing algorithms to recreate depth information, as perceived by the human eye, by mapping the differences between the different perspectives provided by the scene cameras 344. In such embodiments, the controller 390 can execute the image processing algorithms to align and stitch the captured 2D images by matching corresponding points or features in the images, and then calculate depth(s) in the aligned images using triangulation techniques to create a 3D representation of the scene.

In certain embodiments, where multiple scene cameras 344 are fixed at different focal points or planes 414, the varying focus levels of the scene cameras 344 can be used to reconstruct a depth map simulating 3D effects using depth from focus techniques or depth from defocus techniques. For example, in such embodiments, the controller 390 can execute image processing algorithms to measure sharpness in 2D images of the scene captured with varying focus levels, and the measured sharpness can then be utilized to determine depths of pixels in the 2D images. Sharpness can be calculated using any suitable techniques, including gradient-based techniques, Laplacian or Sobel filters, variance of intensity techniques, or the like. By assigning depth values to each pixel, a depth map can be created, which can then be used to the reconstruct a 3D image of the scene.

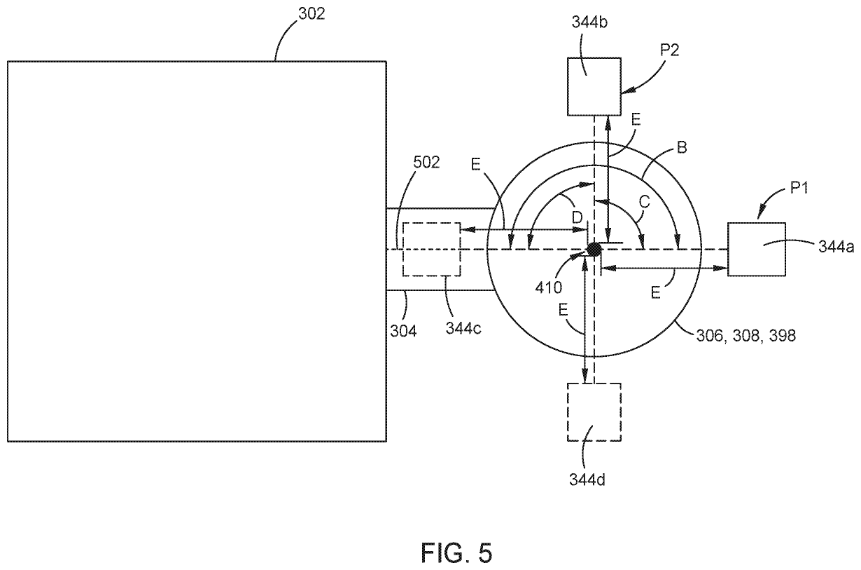

FIG. 5 illustrates a schematic top-down view of an example arrangement of scene camera(s) 344 of the patient interface imaging subsystem 380 relative to the laser delivery head 306 and other components of the ophthalmic surgical system 300, according to certain embodiments of the present disclosure.

In the illustrated embodiments, two scene cameras 344a and 344b are shown, wherein each camera is configured to capture a different perspective or view (i.e., “scene”) of the patient interface 398 and the patient's eye. For simplicity, the patient interface 398 is represented by the same circle as the laser delivery head 306 and the patient attachment module 308 in FIG. 5. During use, the different perspectives provided by the scene cameras 344a and 344b can be displayed to the user on a GUI to assist in the docking process. As noted above, in certain embodiments, the utilization of two or more scene cameras 344 around the patient interface 398 facilitates the capture and generation of 3D images of the patient interface 398 and the patient's eye during docking.

As shown, the first scene camera 344a is disposed at a first position P1 located on a side of the patient interface 398 (and the laser delivery head 306 and patient attachment module 208) opposite the laser delivery extension 304. Generally, the images captured by the scene camera 344a from the position P1 can be referred to as “medial” or “temporal” views of the patient interface 398, since the position P1 will typically correspond to a medial (“inner”) or temporal (“outer”) side of the patient's eye that is being docked and/or operated on. Meanwhile, the second scene camera 344b is disposed at a second position P2 located on a side of the patient interface 398 between the laser delivery extension 304 and the position P1. Generally, the images captured by the scene camera 344a from the position P2 can be referred to as “superior” or “inferior” views of the patient interface 398, since the position P2 will typically correspond to a superior (i.e., top) or inferior (i.e., bottom) side of the patient's eye that is being docked and/or operated on.

As further shown, the position P1 is disposed at an angle B about the major vertical axis 410 relative to a major horizontal axis 502 of the laser delivery extension 304, and an angle C about the major vertical axis 410 relative to the position P2. The position P2 is disposed at an angle D about the major vertical axis 410 relative to the major horizontal axis 502 of the laser delivery extension 304. In certain embodiments, the angle B is between about 90° and about 180°. In certain embodiments, the angle C is between about 1° and about 180°. In certain embodiments, the angle D is between about 1°and about 180°. In specific embodiments, the position P1 is located directly opposite the laser delivery extension 304 relative to the patient interface 398, and the position P2 is located halfway between the laser delivery extension 304 and the position P1 about the major vertical axis 410. In such examples, the angle B is about 180°, the angle C is about 90°, and the angle D is about 90°. Generally, the positions P1 and P2 of the first and second scene cameras 344a and 344b are arranged so as to not interfere with surgeon and/or operating staff access to the patient interface 398 and the patient's eye, while also capturing an unobstructed view of the patient interface 398 (and/or the patient attachment module 208) as it approaches the patient's eye during docking.

In certain embodiments, more or less than two scene cameras 344 can be used to capture additional views of the patient interface 398 and the patient's eye. For example, in certain embodiments, the patient interface imaging subsystem 380 includes a third scene camera 344c (shown in phantom) and/or a fourth scene camera 344d (shown in phantom). In such examples, the third scene camera 344c and fourth scene camera 344d can be configured to capture opposing side, or opposing top or bottom views, respectively, relative to the scene cameras 344a and 344b. In certain embodiments, where three or more scene cameras 344 are used, the scene cameras 344 can be arranged about the major vertical axis 410 at congruent angles relative to each other. In certain embodiments, three or more scene cameras 344 can be arranged about the major vertical axis 410 at noncongruent angles relative to each other.

Generally, each scene camera 344 is disposed at a distance E from the major vertical axis 410 of the laser delivery head 306. In certain embodiments, two or more (or all) of the scene cameras 344 are arranged at a same distance E relative to a major vertical axis 410. In certain embodiments, two or more (or all) of the scene cameras 344 are arranged at different distances E relative to a major vertical axis 410. In certain embodiments, arranging multiple scene cameras 344 at different distances E facilitates the capture and generation of 3D images of the patient interface 398 and the patient's eye during docking.

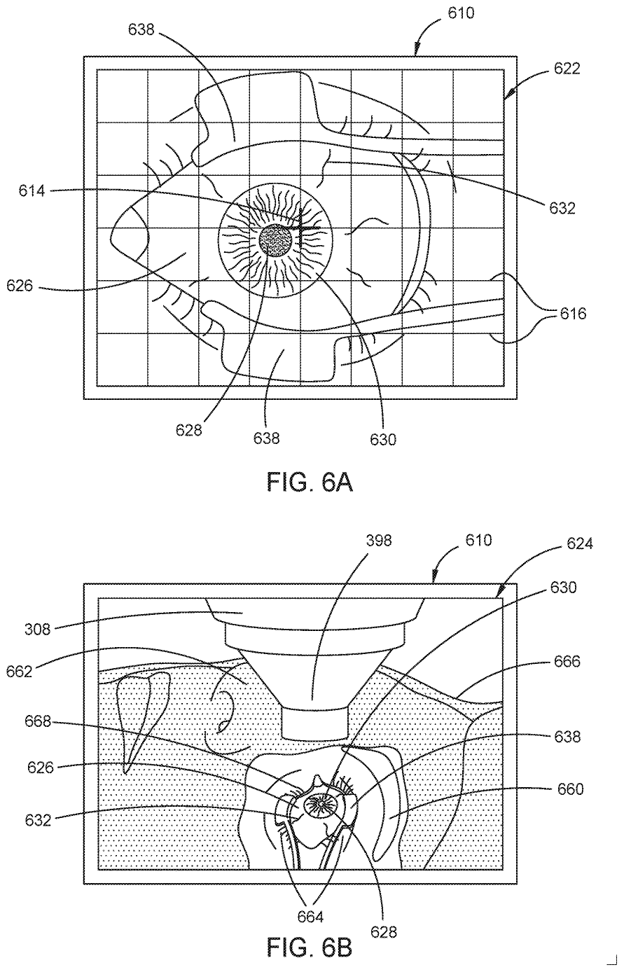

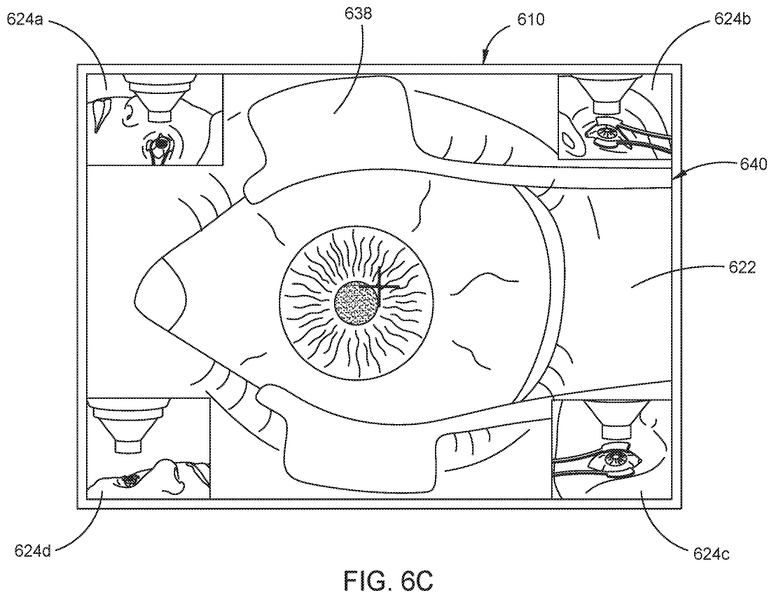

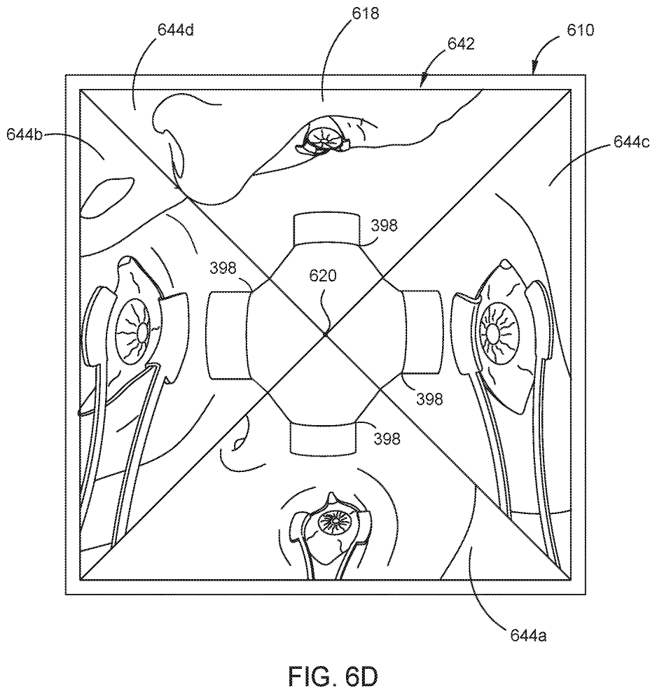

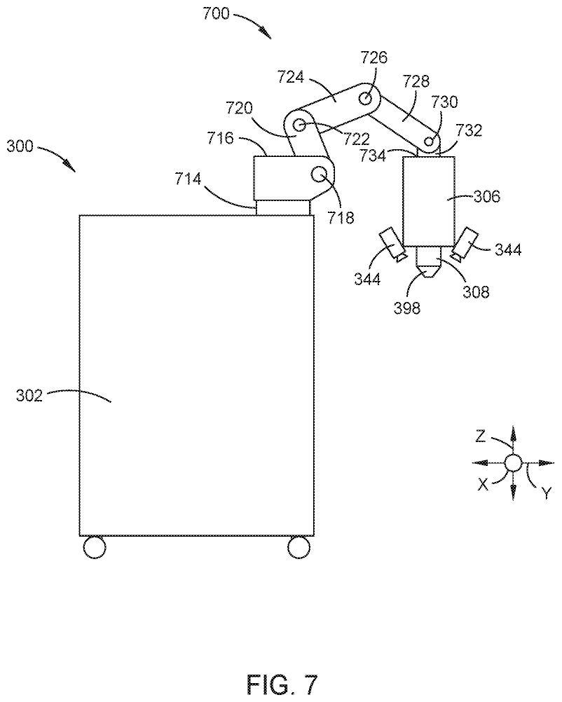

FIGS. 6A-6D illustrate example visuals that can be generated for a user, such as a surgeon or other operating staff member, based on images captured by various components of the ophthalmic surgical system 300 during docking and/or performance of a surgical procedure, according to certain embodiments of the present disclosure. The visuals can be presented to the user on a graphical user interface (GUI) 610 of the ophthalmic surgical system 300, which is generally representative of the GUIs 110 and 210 described above.

FIG. 6A illustrates an example visual 622 of a patient's eye 626 as generated based on images captured by the treatment imaging subsystem 370. As noted above, light is propagated by the treatment imaging subsystem 370 to/from the ophthalmic surgical system 300 through the patient interface 398 and along the imaging optical axis 372. Thus, images captured by the treatment imaging subsystem 370 provide an anterior view of the eye 626.

In certain embodiments, the visual 622 includes a reticle 614 that is vertically aligned with at least one of the imaging optical axis 372 of the treatment imaging subsystem 370, the treatment optical axis 356 of the first laser subsystem 350, the treatment optical axis 358 of the second laser subsystem 352, or the major vertical axis 410 of the laser delivery head 306 in images captured by the treatment imaging subsystem 370. The reticle 614 can be referenced by the user to properly align the laser delivery head 306 with the eye 626 during docking. For example, in certain embodiments, the user can adjust the laser delivery head 306 and/or patient attachment module 308 such that the reticle 614 is aligned with a center of a pupil of the eye 626. In certain embodiments, the reticle 614 is a physical reticle of the laser delivery head 306, patient attachment module 308, and/or ophthalmic surgical system 300. In such embodiments, the reticle 614 can be formed on one or more of the optical devices 354, a lens of the patient interface 398, or another optical device of the ophthalmic surgical system 300 (and aligned with, e.g., the major vertical axis 410 of the laser delivery head 306) that is image-captured by treatment imaging subsystem 370. In certain embodiments, the reticle 614 is a holographic reticle. In certain embodiments, the reticle 614 is a digital reticle generated in the visual 622 by the at least one controller 390.

In certain embodiments, the visual 622 includes a graticule 616, or a grid of lines, that is overlaid onto images captured by the treatment imaging subsystem 370. Similar to the reticle 614, the graticule 616 can be referenced by the user to properly align the laser delivery head 306 with the eye 626 during docking. The graticule 616 can be formed via similar methods to the reticle 614.

FIG. 6B illustrates an example visual 624 as generated based on images captured by a scene camera 344 of the patient interface imaging subsystem 380. In the particular example shown, the images are captured from a scene camera 344 arranged at the temporal position P1 of FIG. 5 and having an oblique orientation relative to the major axis 410 of the laser delivery head 306 and imaging optical axis 372. As a result of this arrangement of the scene camera 344, the images captured thereby provide an oblique and temporal view of the patient interface 398 and the eye 626. However, as noted above, scene cameras 344 of the patient interface imaging subsystem 380 can be arranged at any desired position and angle around the laser delivery head 306 in order to capture a desired view of the patient interface 398 and the eye 626 for display as the visual 624.

In certain embodiments, the visual 624 may further include a reticle 614 and/or a graticule 616, similar to the visual 622 in FIG. 6A. For example, the reticle 614 and/or the graticule 616 may be oriented such that they appear to be in a plane perpendicular to the major axis 410 of the laser delivery head 306 and/or the imaging optical axis 372.

In certain embodiments, a single visual 622 or 624 is displayed on the GUI 610 at a given time to assist the user during the docking process. In certain embodiments, one of each visual 622 and 624 is displayed on the GUI 610 simultaneously. In certain other embodiments, at least one of each visual 622 and 624 is displayed on the GUI 610 simultaneously. For example, in certain embodiments, a single visual 622 and a plurality of visuals 624, corresponding to views provided by different scene cameras 344, are displayed on the GUI 610 simultaneously. In certain embodiments, such visuals 622 and 624 can be displayed on the GUI 610 in a side-by-side or split-screen format. In certain embodiments, the visuals 622 and 624 can be displayed on the GUI 610 in a picture-in-picture arrangement or format, wherein at least the entirety of one visual is overlayed on a portion of another visual. An example of a picture-in-picture format of display is shown in FIG. 6C. Generally, the formatting and other parameters for display of the visuals 622 and 624 on the GUI 610 can be predefined, or preset, by the user prior to performing a docking process and/or a surgical procedure via a user input device of the ophthalmic surgical system 300. Where a plurality of visuals are displayed simultaneously, the combination of visuals can be referred to as “a display” of visuals.

With reference now to FIG. 6C, a first display format 640 for a combination of visuals is shown on the GUI 610. A background or primary display of the display format 640 includes the centrally-displayed visual 622, and a foreground of the display format 640 includes four visuals 624a-d overlayed onto four corners of the visual 622 to form a picture-in-picture format. The visual 622 provides an anterior view of the eye 626 based on images captured by the treatment imaging subsystem 370, while the visuals 624a-d provide oblique views from different sides of the patient interface 398. Visual 624a corresponds to the visual 624 in FIG. 6B, and thus shows an oblique and temporal view of the patient interface 398; visual 624b shows an oblique and inferior view of the patient interface 398; visual 624c shows an oblique and superior view of the patient interface 398; and visual 624d shows an oblique and medial view of the patient interface 398. Please note that the arrangement in FIG. 6C is only exemplary, and any arrangement and/or order of the visual 622 and/or visuals 624a-d is contemplated.

Further, although four visuals 624a-d are shown in FIG. 6C, in certain embodiments, less than four visuals 624, or more than four visuals 624, can be overlayed onto the visual 622. For example, in certain embodiments, only one or two or three visuals 624 are overlayed onto the visual 622; in certain embodiments, five or six or more visuals 624 are overlayed onto the visual 622. In embodiments where two visuals 624 are overlayed onto the visual 622, the visuals 624 can include at least one of a superior view and an inferior view, and/or may include at least one of a medial view and a temporal view.

Generally, the visuals 624 can be overlayed onto the visual 622 at positions along a periphery, or outer edge, of the visual 622; however, any suitable positions on the visual 622 are contemplated for overlaying the visuals 624. In certain embodiments, the overlayed visuals 624 can be offset from the outer edge of the visual 622 by a defined distance (e.g., in pixels).

In still further embodiments, a visual 624 can be arranged as the background of the display format 640, as opposed to the visual 622. In such embodiments, the visual 622 can be included in the foreground and overlayed onto the visual 624, or not displayed at all. In certain embodiments, a user can dynamically cycle between which visual 622 or 624 is displayed in the background of the display format 640, and which visual(s) 622 and/or 624 are displayed in the foreground of the display format 640, prior to or during the docking process. For example, in certain embodiments, the user can provide input to the ophthalmic surgical system 300 via a user input device, which can cause the background and foreground visuals to change and/or exchange.

With reference now to FIG. 6D, a second display format 642 for a combination of visuals is shown on the GUI 610. The display format 642 in FIG. 6D can be referred to as a “panoramic” or “surrounding” display format. In FIG. 6D, images 644a-d from a plurality of scene cameras 344 are captured, processed (e.g., by controller 390), and “stitched” together (e.g., by controller 390 using image processing algorithms) to form a combined “surrounding” visual 618 of the patient interface 398 and the eye 626 for display on the GUI 610 during docking. Here, “stitching” can refer the process of combining multiple images with overlapping, or non-overlapping, fields-of-view to create a single, composite image, such as a panorama. In certain embodiments, a stitched visual includes a seamless composite image. In certain embodiments, a stitched visual includes a segmented composite image.

In the example of FIG. 6D, the stitched images 644a-d correspond to the images utilized for the visuals 624a-d of FIG. 6C, as captured by at least four scene cameras 344 with four different views from different sides of the patient interface 398 (and/or the patient attachment module 308). However, less than four different views, or more than four different views, can be utilized to form the stitched image in the display format 642. For example, in certain embodiments two or three views can be stitched together to form the display format 642. In certain embodiments, the stitched images 644 a-d can form up to a 360°object view around the patient interface 398 and eye 626. For example, the stitched images 644a-d can form a combined image capturing up to a 360° rotation around the patient interface 398 and eye 626, with an “outside-in” perspective. In some examples, the stitched images 644a-d can form a 240° object view, a 180° object view, a 90° object view, or a 45° object view around the patient interface 398 and eye 626. Note that for purposes of simplicity and clarity, only four images 644a-d are illustrated in FIG. 6D; however, more than four images 644 may need to be used to form a seamless, up to a 360°, object view of the patient interface 398 and eye 626. In some examples, the stitched images 644a-d may present a view that is different from any of the other cameras, such as the four scene cameras 344. In some embodiments, the view presented may include a view horizontally aligned with a distal end of the patient interface 398, horizontally aligned with a top surface of a cornea of the eye 626, and/or horizontally aligned with the iris or other internal feature of the eye 626.

In the illustrated embodiments, four images 644a-d are arranged and stitched around a central focal point 620 of the visual 618, which corresponds to the patient interface 398. However, any suitable arrangement of images captured by the scene cameras 344 can be utilized to stitch the visual 618. For example, the central focal point 620 of the visual 618 may be the eye 626, or any other feature.

In certain embodiments, the images 644a-d utilized for the display format 642 can be captured by scene cameras 344 with different focal lengths relative to the patient interface 398. However, based on a determined focal length of a scene camera 344, the controller 390 can transform, or warp, the corresponding images from the scene camera 344 to seamlessly “stitch” its images with images from other scene cameras 344 with different focal lengths when generating the display format 642 to create a coherent or consistent visual 618.

In certain embodiments, the ophthalmic surgical system 300 is configured to recognize features in images captured via the treatment imaging subsystem 370 and/or the patient interface imaging subsystem 380. For example, in certain embodiments, the images captured by the treatment imaging subsystem 370 and/or the patient interface imaging subsystem 380 can be transmitted to, and processed by, the at least one controller 390, which can identify one or more features in the captured images. Such features can include facial features of the patient including, but not limited to, one or both eyebrows 660, a nose 662, eyelashes 668, or eyelids 664, and/or features of the eye 626, such as a pupil 628, an iris 630, and/or vasculature 632 of the eye 626. In certain embodiments, the one or more features can include non-facial features, such as a surgical drape 666 disposed around the eye 626, and/or a speculum 638 utilized to hold the eyelids 664 open. In certain embodiments, the identified features are highlighted, or called out, in a visual presented to the user on the GUI 610 of the ophthalmic surgical system 300 to assist in the docking process.

In certain embodiments, using the feature recognition function, the at least one controller 390 can determine an orientation of the eye 626 by mapping the recognized features relative to each other and to the general field-of-view in images captured by the treatment imaging subsystem 370 and/or the patient interface imaging subsystem 380. For example, the orientation of the eye 626 can be determined based on the relative positions of the pupil 628, the iris 630, and/or the vasculature 632, as related to each other and/or other facial features. In certain embodiments, a center of the pupil 628 and/or axis of the eye can be identified via the feature recognition function, and then highlighted relative to a reticle (e.g., reticle 614) of the ophthalmic surgical system 300. In further embodiments, using the feature recognition function, the at least one controller 390 can identify which eye 626 (e.g., whether the eye 626 is a left eye or a right eye) of the patient is being docked by mapping the recognized features relative to each other and to the general field-of-view in images captured by the treatment imaging subsystem 370 and/or the patient interface imaging subsystem 380. For example, the identification of the eye 626 can be performed based the relative positions of the nose 662, the eye 626, and/or an eyebrow 660, as related to each other and/or other facial or non-facial features. Where the patient's head is covered by the surgical drape 666, the eye 626 can be identified based on the relative orientation and/or position of the speculum 638 relative to the eye 626. In certain embodiments, the identified eye is highlighted, called out, or otherwise indicated in a visual presented to the user on the GUI 610 of the ophthalmic surgical system 300 to assist in the docking process. For example, an indication of whether the eye is a left eye or a right eye of the patient can be included in a visual generated and displayed on the GUI 610 during the docking process. Information related to the identified eye can then be stored by ophthalmic surgical system 300 for later use.

In certain embodiments, the feature recognition of ophthalmic surgical system 300 can be utilized to facilitate completely, or at least partially, automated docking of a patient's eye. For example, in certain embodiments, the laser delivery extension 304 of the ophthalmic surgical system 300 includes an automated robotic arm. FIG. 7 illustrates an example robotic arm 700 that can be used with the ophthalmic surgical system 300 to provide automated docking, according to certain embodiments of the present disclosure.

As noted above, in certain embodiments, the robotic arm 700 is representative of, or a component of, the laser delivery extension 304. Generally, the robotic arm 700 is configured to move the laser delivery head 306, the patient attachment module 308, and the patient interface 398 into alignment with an eye of patients of various sizes. The robotic arm 700 may be understood with respect to X, Y, and Z direction, where the Z direction is substantially (e.g., within 2 degrees of) parallel to the direction of gravity and the X and Y directions are substantially (e.g., within 2 degrees of) perpendicular to the Z direction and to one another. The robotic arm 700 is configured to move the laser delivery head 306 in the X, Y, and Z directions, as well as one or more rotational degrees of freedom, such as rotation about an axis substantially (e.g., within 2 degrees of) parallel to the X, Y, and/or Z direction.

The robotic arm 700 can include a rotational joint 714 coupled to a base 716. The robotic arm 700 can be mounted to the chassis 302, such as by the rotational joint 714. The base 716 is coupled by an elbow joint 718 to a link 720. An elbow joint 722 couples the link 720 to a link 724. An elbow joint 726 couples the link 724 to a link 728. An elbow joint 730 couples the link 728 to a link 732. The link 732 may be coupled by a rotational joint 734 to the laser delivery head 306. Each of the illustrated joints 714, 718, 722, 726, 730, and 734 can have a corresponding actuator for inducing movement of the joint. In certain embodiments, the robotic arm 700 can have at least five (5) degrees of freedom (DOF). For example, in certain embodiments, the illustrated robotic arm 700 has six (6) DOF. In certain embodiments, the robotic arm 700 can be embodied as a commercially available serial robotic arm. The robotic arm 700 can also be implemented as linear actuators, such as linear actuators implementing movements in the X, Y, and Z directions, as well as one or more rotational actuators inducing rotation about one or more of the X, Y, and Z axes. For example, the robotic arm 700 may be embodied as gantry.

The actuators of the robotic arm 700 can be coupled to a controller, such as the at least one controller 390 described above. In certain embodiments, the at least one controller 390 can receive images from the treatment imaging subsystem 370 and/or the patient interface imaging subsystem 380 (e.g., images captured by the scene cameras 344), and/or one or more additional cameras of the robotic arm (not shown) or other components of the ophthalmic surgical system, in order to estimate a three-dimensional position of the patient's head and/or the patient's eye, and then activate the robotic arm 700 to automatically position the patient interface 398 at or within a threshold distance of the patient's head during docking. Images from the treatment imaging subsystem 370 and/or the patient interface imaging subsystem 380 can further be used by the at least one controller 390 to automatically perform fine adjustments to the position of the patient interface 398 to dock the laser delivery head 306 to the patient's eye. Alternatively, the laser delivery head 306 can incorporate actuators that are controlled to automatically perform fine adjustments of the laser delivery head 306 and/or the patient interface 398 based on one or more images.

The position of the patient interface 398 itself can be determined by sensing a kinematic state of the robotic arm 700 using sensors incorporated into the joints 714, 718, 722, 726, 730, 734 or elsewhere in the robotic arm 700. Alternatively or additionally, the position of the patient interface 398 may also be determined based on images received by the at least one controller 390.

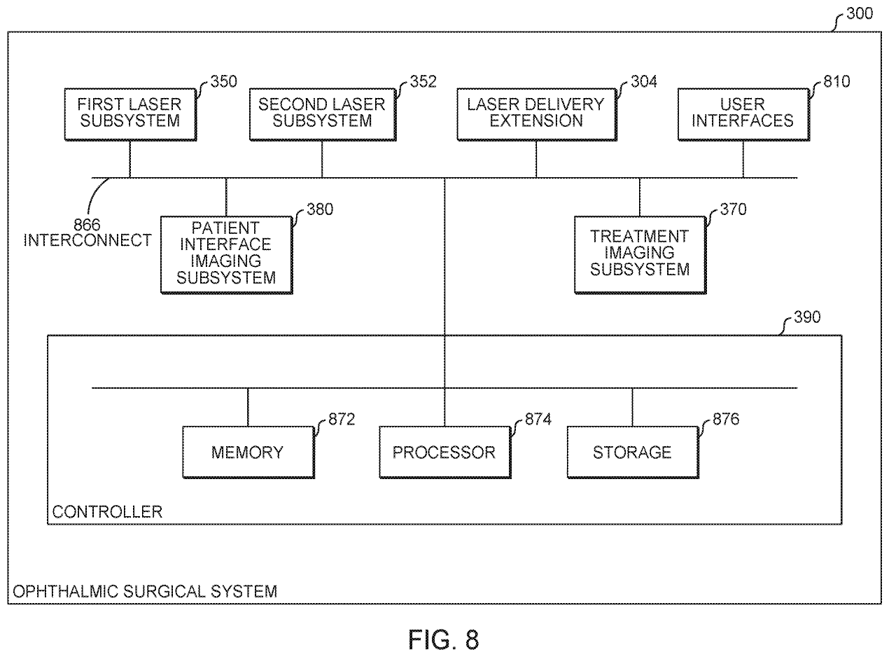

FIG. 8 illustrates a schematic diagram of various electronic components of the ophthalmic surgical system 300, according to embodiments disclosed herein.

The ophthalmic surgical system 300 includes the controller 390 for controlling operations of, for example, the first laser subsystem 350, the second laser subsystem 352, the treatment imaging subsystem 370, the patient interface imaging subsystem 380, the laser delivery extension 304 (e.g., the robotic arm 700), user interfaces 810, and/or other components of the ophthalmic surgical system 300.

In the illustrated diagram, the user interfaces 810 can include any user interfaces of the ophthalmic surgical system 300. For example, the user interfaces 810 can include the GUIs 110 and 210, the joystick 340, footswitches 124 and 224, display devices, keyboards, touchpads, switches, levers, buttons, or other user input and/or output devices from which the ophthalmic surgical system 300 may receive user input for controlling functions thereof, and/or provide output to the user.

The controller 390 includes a processor 874, a memory 872, and/or a storage 876. The processor 874 (e.g., control circuitry) is configured to retrieve and execute programming instructions stored in the memory 872. Similarly, the processor 874 may retrieve and store application data residing in the memory 872. An interconnect 866 transmits programming instructions and application data, among the processor 874, memory 872, storage 876, etc. The processor 874 may include a single CPU, multiple CPUs, a single CPU having multiple processing cores, and the like. The memory 872 may be random access memory, and the storage 876 may be a disk drive. Moreover, the memory 872 and/or storage 876 may be any type of a readily available memory, such as random access memory (RAM), read only memory (ROM), floppy disk, hard disk, solid state, flash memory, magnetic memory, or any other form of digital storage, local or remote.

In the embodiments of FIG. 8, the processor 874 may include an integrated circuit capable of performing logic functions. In this manner, the processor 874 is in the form of a standard integrated circuit package with power, input, and output pins. In certain embodiments, the processor 874 may perform specific control functions targeted to a specific device, such as one or more scene cameras 344 of the patient interface imaging subsystem 380, user interfaces 810, etc. In certain embodiments, the processor 874 is a microprocessor. In certain embodiments, the processor 874 is not a programmable microprocessor, but instead is a special purpose controller configured to control different components that perform different functions. In some embodiments, the processor 874, memory 872, and/or storage 876 may be implemented as a single standalone chip.

The present disclosure, in certain embodiments, provides patient interface imaging systems for use during patient docking procedures for ophthalmic surgical procedures. The systems enable a surgeon, or other operating staff, to observe and control the docking process from an ergonomic position, thereby facilitating docking of a patient's eye without causing strain or fatigue to the surgeon before performance of the surgical procedures. Further, the patient interface imaging systems described herein provide the surgeon with multiple enlarged and dynamic views of the patient's eye and patient interface, thereby facilitating precise and accurate docking.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. The term “about,” when used in conjunction with a numerical value, refers to plus or minus 5%, or plus or minus 10%, of the numerical value. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the subject matter have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the disclosed subject matter, but instead as merely intended to highlight alternative or additional features that may or may not be utilized in a particular example of the subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosed to the specific examples and examples described herein, but rather to also cover all modification and alternatives coming with the true scope and spirit of the subject matter.

What is claimed is:

1. An ophthalmic surgical system, comprising:
a chassis comprising a laser source configured to generate a laser;
a laser delivery head adjustably attached to the chassis and configured to deliver the laser to an eye;
a patient interface, the patient interface comprising at least a first component attached at a distal end of the laser delivery head and configured to facilitate docking of the laser delivery head onto the eye for delivery of the laser to the eye; and
a patient interface imaging system, comprising:
a first scene camera configured to capture images of the patient interface during docking of the laser delivery head onto the eye for display to a user on a graphical user interface (GUI); and
a second scene camera configured to capture images of the patient interface during docking of the laser delivery head onto the eye, wherein:
the first scene camera and the second scene camera are arranged such that optical axes of each of the first scene camera and the second scene camera are oriented at oblique angles relative to a major axis of the laser delivery head; and
the first scene camera and the second scene camera are arranged about 90° or more from each other about the major axis of the laser delivery head.

2. The ophthalmic surgical system of claim 1, wherein at least one of the first scene camera or the second scene camera comprises a complementary metal oxide semiconductor (CMOS) camera configured to transmit image data via Mobile Industry Processor Interface (MIPI) protocols.

3. The ophthalmic surgical system of claim 1, wherein at least one of the first scene camera or the second scene camera comprises an infrared (IR) camera.

4. The ophthalmic surgical system of claim 3, wherein the IR camera comprises a red-green-blue-infrared (RGB-IR) camera.

5. The ophthalmic surgical system of claim 1, wherein at least one of the first scene camera or the second scene camera comprises molded polymer optics.

6. The ophthalmic surgical system of claim 1, wherein at least one of the first scene camera or the second scene camera comprises telecentric optics.

7. The ophthalmic surgical system of claim 1, wherein the patient interface further comprises a second component separate from the first component and configured to be placed onto the eye prior to or during docking of the laser delivery head onto the eye, and wherein the first component is configured to engage with the second component during docking of the laser delivery head onto the eye.

8. The ophthalmic surgical system of claim 1, further comprising:
a controller in data communication with the first scene camera and the second scene camera, the controller configured to process images captured by the first scene camera and the second scene camera to generate a display format simultaneously presenting the images captured by the first scene camera and the second scene camera on the GUI.

9. The ophthalmic surgical system of claim 8, wherein the display format comprises a stitched composite visual combining the images captured by the first scene camera and the second scene camera.

10. The ophthalmic surgical system of claim 8, further comprising:
a treatment imaging system, the treatment imaging system configured to capture images of the eye through the patient interface, wherein:
the controller is further configured to process the images captured by the treatment imaging system to generate the display format comprising a picture-in-picture arrangement of the images captured by at least one of the first scene camera and the second scene camera and the images captured by the treatment imaging system.

11. An ophthalmic surgical system, comprising:
a laser delivery device configured to deliver a laser to an eye of a patient;
a patient interface attached to the laser delivery device and configured to facilitate docking of the laser delivery device onto the eye for delivery of the laser to the eye; and
a patient interface imaging system, the patient interface imaging system comprising:
a plurality of scene cameras configured to capture images of the patient interface during docking of the laser delivery device onto the eye for display on a graphical user interface (GUI), wherein each of the plurality of scene cameras is arranged such that an optical axis of the scene camera is oriented at an oblique angle relative to a major axis of the laser delivery device.

12. The ophthalmic surgical system of claim 11, wherein:
the plurality of scene cameras comprises at least a first scene camera and a second scene camera arranged about 90° or more from each other about the major axis of the laser delivery device.

13. The ophthalmic surgical system of claim 11, wherein at least one of the plurality of scene cameras comprises a complementary metal oxide semiconductor (CMOS) camera configured to transmit image data via Mobile Industry Processor Interface (MIPI) protocols.

14. The ophthalmic surgical system of claim 11, wherein at least one of the plurality of scene cameras comprises an infrared (IR) camera.

15. The ophthalmic surgical system of claim 14, wherein the IR camera comprises a red-green-blue-infrared (RGB-IR) camera.

16. The ophthalmic surgical system of claim 11, wherein at least one of the plurality of scene cameras is adjustably attached in at least one of an orientation or a position about a laser delivery head of the laser delivery device.

17. The ophthalmic surgical system of claim 11, further comprising:
a controller in data communication with the plurality of scene cameras, the controller configured to process images captured by the plurality of scene cameras to generate a display format simultaneously presenting the images captured by the plurality of scene cameras for display on the GUI.

18. The ophthalmic surgical system of claim 17, wherein the display format comprises a stitched composite visual combining the images captured by the plurality of scene cameras.

19. The ophthalmic surgical system of claim 17, further comprising:
a treatment imaging system, the treatment imaging system configured to capture images of the eye through the patient interface, wherein:
the controller is further configured to process the images captured by the treatment imaging system to generate the display format comprising a picture-in-picture arrangement of the images captured by the plurality of scene cameras and the images captured by the treatment imaging system.

20. The ophthalmic surgical system of claim 17, wherein:
the controller is further configured to identify the eye as a left eye or a right eye of the patient based on at least one of a facial feature of the patient or a non-facial feature in the captured images, and to further provide an indication of the identified eye in the captured images.
